(19) United States
(12) Patent Application Publication
(10) Pub. No.: US 2021/0268129 A1
(43) Pub. Date: Sep. 2, 2021

(54) Theranostic Radiophotodynamic Therapy Nanoparticles

(71) Applicant: The Governers of the University of Alberta, Edmonton (CA)

(72) Inventors: Deepak Dinakaran, Edmonton (CA); Ronald Moore, Edmonton (CA); John Lewis, Edmonton (CA); Ravin Narain, Edmonton (CA); Piyush Kumar, Edmonton (CA); Nawaid Usmani, Edmonton (CA)

(21) Appl. No.: 17/255,424

(22) PCT Filed: Jun. 20, 2019
(86) PCT No.: PCT/CA2019/050866

§ 371 Date: Dec. 22, 2020

Related U.S. Application Data
(60) Provisional application No. 62/688,481, filed on Jun. 22, 2018.

(51) Int. Cl. A61K 49/04; A61K 41/00; C09K 11/02; C09K 11/77

(52) U.S. Cl. CPC A61K 49/0423; A61K 41/0071; B82Y 5/00; C09K 11/7719; C09K 11/02

(57) ABSTRACT

A nanoparticle includes a nanocarrier encapsulating a nanoscintillator capable of emitting light upon exposure to radiation; a photosensitizer capable of absorbing the light from the nanoscintillator to generate singlet oxygen species; and optionally, one or more diagnostic agents, therapeutic agents, or a combination thereof.

### CROSS-REFERENCE TO RELATED APPLICATIONS

This application claims the benefit of U.S. Provisional Patent Application Ser. No. 62/688,481, filed Jun. 22, 2018, the entirety of which is incorporated herein by reference (where permitted).

### FIELD OF THE INVENTION

The present invention relates to theranostic radiophotodynamic therapy nanoparticles, pharmaceutical compositions comprising same, and methods of preparing and using same.

### BACKGROUND OF THE INVENTION

In cancer treatment, the primary modalities are generally divided into surgical management, radiotherapy, and chemotherapy. To manage cancer effectively, clinicians use one or a combination of these treatment modalities. Since each treatment type has distinct advantages and disadvantages, the clinicians and patients must decide which modality of management is the best option in the context of the patient's disease.

Radiotherapy has been used successfully to treat tumors by directing ionizing X-rays at the tumor to cause direct or indirect DNA damage. This causes the cell to lose its proliferative potential or undergo apoptosis. It is used alone and in combination with the other modalities to treat tumors in both palliative and curative settings. In prostate cancer, it is one of the primary modalities of cure available to patients, and provides comparable outcomes to surgical management of the disease without the risks of an invasive operation. However, radiotherapy has limitations in the form of side effects and long-term morbidity related to the dose of radiation delivered to normal structures around the tumor, which inadvertently also receive radiation. Modern radiotherapy involves precise calculation of the doses to the target and neighboring normal organs at risk to ensure that the target receives an adequate dose, while not exceeding set tolerances to normal organs.

Advances in radiotherapy have allowed radiation oncologists to increase the radiation dose to the cancerous regions safely, while still maintaining the dose received by normal tissues at acceptable levels. With these higher doses, better oncologic outcomes have been achieved, without causing significantly higher toxicity. However, there are limits with what conformal, intensity-modulated radiotherapy can achieve with a traditional radiation machine. Higher precision technologies (for example, proton therapy, gamma knife, cyber knife) are available to achieve even higher conformity, but such techniques are limited in their cost effectiveness and in how much dose they can safely deliver to the tumor before the toxicity of treatment outweighs the benefits. In prostate radiotherapy, the dose delivered to the prostate has been increased safely from 60 Gy in the past to 78 Gy in modern standard of care, while maintaining toxicity levels at about the same rate. There is evidence to show that even higher doses of radiation can improve oncologic outcomes, but the resulting toxicity makes higher doses unfeasible in widespread practice.

Photodynamic therapy (“PDT”) has been implemented in clinical medicine in various indications, including cancer care, dermatologic conditions, ophthalmologic conditions, and infection treatment. Its mechanism of action is via excitation of a photosensitizer that reacts with surrounding molecules to form radical species. The majority of its cytotoxicity is conferred through transferring its energy from its excited form to oxygen, and forming singlet oxygen. This form of oxygen species is short-lived and highly reactive, which means it causes significant cellular damage only to a localized area. In clinical use, PDT is conducted by administering a drug which can act as a photosensitizer when activated with a laser light source at the target site. This confers significant cytotoxicity and necrosis to the diseased tissue, but spares toxicity to adjacent healthy tissue. It has also been of interest in cancer research for many years due to its remarkable cell-killing potential, and low toxicity of the drug by itself outside the field of light irradiation. Recently, it is also suspected to potentiate immunogenic response to tumors since the mechanism of cellular damage facilitates antigen presentation to immune system cells. However, it has failed to gain widespread clinical use due to its dependence on visible wavelength light. This has a limited penetrance into tissues in the body, and is often only limited to less than 1 centimeter. This means only surface or endoluminal tumors can be treated, or otherwise an invasive strategy with light catheters is needed to deliver light into deeper tumors. The distribution of light irradiation dose through tissue (i.e., dosimetry) has also been challenging to quantify, which makes it hard to standardize PDT for therapeutic effect from patient to patient, and has contributed to its limited clinical use currently.

Radiation activated photodynamic therapy (radioPDT) combines the advantages of radiotherapy with PDT. US 2007/0218049 to Chen and Zhang described using ionizing radiation to induce luminescence in a particle, and the use of the luminescence for activating compounds capable of photodynamic therapy. However, the need to monitor the therapy delivered in order to ensure confidence on the level of therapy delivered, and the characteristics of a biocompatible or targeted nanoparticle were not addressed. Clement et al. (2016) used high energy X-rays to test radioPDT and quantified the quantum yield of singlet oxygen per dose of radiation, allowing for radiation dose modeling to determine how much therapeutic effect might be expected. However, the particle used was not assessed for stability of the conjugation or its biocompatibility. Fang et al. (2015) described simultaneously imaging and treating with a PDT capable agent, but their imaging and treating technique relies upon direct access to the target with a light source and would be unsuitable for deep seated tumors. Shi et al. (2014) described a biocompatible fullerene-based nanoparticle having a favorable size, MRI imaging characteristics, and potential for light PDT and radiofrequency thermal therapy based treatment. However, the method of tumor targeting is impractical in humans, and the particle exhibits limited penetration in tissue. Tang et al. (2015) described a nanoscintillator compound encapsulated in silica in combination with a photosensitizer to generate PDT for use as a CT contrast agent. However, the particle exhibited shortcomings with respect to uncertain biocompatibility and circulation, limited diagnostic capability, and failure to quantify singlet oxygen yield which is the main cytotoxic agent generated from PDT. Zou et al. (2014) described oversized nanoparticles (>1000 nm) stabilized in a toxic medium (dimethyl-sulfoxide). Radiosensitizers are available for clinical use, such as cytotoxic chemotherapy with fluorpyrimidine analogs, platinum-based agents, and bio-reductively activated nucleotide analogs. Such agents augment the double strand DNA brakes caused by radiation, but do not have any function in performing PDT. Accordingly, there is a need for improved radioPDT materials, particularly in the treatment of diseases such as cancer.

### SUMMARY OF THE INVENTION

The present invention relates to theranostic radiophotodynamic therapy nanoparticles, pharmaceutical compositions comprising same, and methods of preparing and using same.

In one aspect, the invention comprises a nanoparticle comprising a nanocarrier encapsulating a nanoscintillator capable of emitting light upon exposure to radiation; a photosensitizer capable of absorbing the light from the nanoscintillator to generate singlet oxygen species; and optionally, one or more diagnostic agents, therapeutic agents, or a combination thereof.

In one embodiment, the nanocarrier comprises a hydrophobic compound and a hydrophilic compound. In one embodiment, the hydrophobic compound comprises poly lactide co-glycolide acid. In one embodiment, the hydrophilic compound comprises polyethylene glycol, or polyethylene glycol bound to an antibody.

In one embodiment, the nanoscintillator comprises a radioluminescent material doped with a dopant selected from terbinium, yttrium, sodium, or cerium. In one embodiment, the amount of dopant ranges from about 0% to about 50%. In one embodiment, the nanoscintillator comprises lanthanum fluoride doped with 10% cerium. In one embodiment, the nanoscintillator is hydrophobic. In one embodiment, the nanoscintillator is operably associated with the photosensitizer by physical proximity or chemical linkage.

In one embodiment, the photosensitizer is selected from a thiocyanate, vertiporfin, hypocrellin A, hypocrellin B, or protoporphyrin IX. In one embodiment, the photosensitizer is hydrophobic.

In one embodiment, the nanoparticle has a size ranging from about 30 nm to about 150 nm. In one embodiment, the nanoparticle has a size ranging from about 100 nm to about 120 nm.

In another aspect, the invention comprises a pharmaceutical composition comprising the above nanoparticle and a pharmaceutically acceptable carrier.

In another aspect, the invention comprises a method of treating, preventing, or ameliorating a disease in a subject, comprising the steps of: a) administering to the subject an effective amount of the above nanoparticle, or the above pharmaceutical composition; and b) applying radiation to the subject.

In one embodiment, the nanoparticle or the pharmaceutical composition is administered to the subject intravenously. In one embodiment, the radiation comprises X-rays. In one embodiment, the disease is selected from cancer, a dermatological condition, an infection, macular degeneration, Barrett's esophagus, a deep abdominal abscess, or a condition requiring cell cytotoxicity. In one embodiment, the nanoparticle comprises one or more diagnostic agents, therapeutic agents, or a combination thereof for release in a localized area following application of the radiation.

In yet another aspect, the invention comprises use of the above nanoparticle, or the above pharmaceutical composition to treat, prevent, or ameliorate a disease in a subject. Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1A is a transmission electron microscopy (TEM) image of 10% Cerium-doped lanthanum (III) fluoride crystals. FIGS. 1B-C are transmission electron microscopy images showing a sampling of size measurements of the nanoscintillators. FIG. 1D is a graph of particle concentration versus hydrodynamic diameter of the nanoscintillators as measured by DLS after a 1000× dilution, showing a measured particle concentration of (9±1)×10¹¹ particles per mL. FIG. 1E shows a UV-VIS absorption spectrum of the nanoscintillators showing absorbance peaks characteristic of 10% cerium doped lanthanum fluoride. FIG. 1F shows a fluorescence emission spectrum of the nanoscintillators. FIG. 1G shows the luminescence emission spectra under radiation 250 nm excitation for the nanoscintillators.

FIGS. 2A-D show LaF₃:Ce nanoscintillators synthesized in aqueous vs. organic media under TEM (FIG. 2A). Differences in relative intensities of absorption peaks are demonstrated (FIG. 2B), along with fluorescence spectra (FIG. 2C). The XRD profiles of the aqueous phase and the organic phase synthesized LaF₃:Ce nanoscintillators are shown (FIG. 2D).

FIG. 3A is a graph showing nanoscintillators CT attenuation measured with a Varian TruBeam STx cone beam imager showing signal enhancement of 0.0957 Houndsfield(H.U.)/ppm. FIG. 3B is a graph showing MM signal inversion time analysis with a Philips 3 Tesla MRI measuring a T1 relaxivity constant of 1.122×10⁻⁷ ms/ppm. FIG. 3C is a graph showing MM signal inversion time analysis with a Philips 3 Tesla MRI measuring a T2 relaxivity constant of 2.398×10⁻⁷ ms/ppm.

FIG. 4A is a schematic diagram showing the chemical structure of polyethylene glycol-polylactic acid-co-glycolic acid (PEG-PLGA). FIG. 4B is a schematic diagram showing a method of nanoparticle formation with payload encapsulation.

FIGS. 5A-C are TEM images showing morphology (i.e., shape and size distribution) of PEG-PLGA nanocarriers when empty or unloaded (FIG. 5A), loaded with nanoscintillators (FIG. 5B), and loaded with nanoscintillators and photosensitizer (FIG. 5C). The corresponding UV-Vis spectroscopy for each condition is shown in FIGS. 5D-F, and the concentration as a function of size via dynamic light scattering (DLS) is shown in FIGS. 5G-I.

FIGS. 6A-C show PPIX and nanoscintillator encapsulated PEG-PLGA microspheres are shown under TEM with bright field (FIG. 6A), lanthanum element mapping (FIG. 6B), and oxygen element mapping mode (FIG. 6C).

FIGS. 7A-D show the stability as measured by NP size demonstrated in human plasma (FIG. 7A) and human serum (FIG. 7B) at 37 degrees Celsius. The release kinetics of PPIX from the NP is shown in water at room temperature and physiologic temperature over 72 hours (FIG. 7C) and DMEM with 10% FBS (FIG. 7D) at physiologic temperature.

FIG. 8 shows X-ray images of a chick chorioallantoic membrane (CAM) model with HT1080 tumors implanted on day 10 with X-ray imaging studies done on day 15 and 16. The CAM was sequentially imaged immediately after IV injection of NSC and 12 min later to demonstrate intra-tumoral accumulation over time (circle).

FIGS. 9A-C show the mouse phantom used for CT attenuation assessment (FIG. 9B) and via cross-sectional view from CT scanning the phantom with water, omnipaque, and NSC (top, middle, and bottom of FIG. 9A). The measured Hounsfield units are plotted against concentration to demonstrate their efficiency as a CT contrast agent (FIG. 9C).

FIG. 10 shows results of fluorescence spectroscopy of radioPDT NP with 200 nm excitation. Control conditions of nanoscintillator-only NP and PPIX alone are shown for reference. A fluorescence peak at 620 nm (PPIX emission maxima) is apparent only in the functional radioPDT NP with both NSC and PPIX for FRET.

FIG. 11 shows the results of singlet oxygen yield production measured after 10.6 Gy of RT was delivered. Control conditions of NP containing the scintillator only and photosensitizer are shown for comparison. The NP were dosed at 1.0e12 particles/ml and showed no difference in detectable singlet oxygen yield under these conditions in normoxia (20% O₂) and hypoxia (0.45% O₂).

FIG. 12 is a graph showing ¹O₂ yield as measured by SOSG probe with NSC and PPIX loaded PEG-PLGA nanocarriers (radioPDT NP) under 2 Gy of RT delivered in differing hypoxia conditions. NSC loaded PEG-PLGA nanocarriers (SCN NP) is provided as a control.

FIGS. 13A-C show in vitro cellular toxicity radioPDT NP and its constituents in prostate cancer lines PC3 (FIG. 13A) and DU145 (FIG. 13B), and skin fibroblast line GM38 (FIG. 13C).

FIGS. 14A-F show cellular uptake and localisation study in PC3 cells. Images were taken after 24 hrs, after incubation with (FIG. 14A) PC3 untreated cells (left panel) (FIG. 14B) PEG-PLGA empty NPs treated PC3 cells (10⁸ NPs/mL) (left panel) (FIG. 14C) PEG-PLGA+NSC NPs treated PC3 cells (10⁸ NPs/mL) (left panel) (FIG. 14D) PEG-PLGA+NSC+PPIX NPs treated PC3 cells (10⁸ NPs/mL) (left panel) (FIG. 14E) PEG-PLGA+NSC+PPIX+dye NPs treated PC3 cells (10⁸ NPs/mL) (left panel) (FIG. 14F) Z-stack of PEG-PLGA+NSC+PPIX+dye NPs treated PC3 cells (10⁸ NPs/mL) (right panel) Images clearly demonstrate nanospheres with TT1 dye are internalized by the cells.

FIGS. 15A-D show cytotoxicity of radioPDT NP compared to control conditions under UV light irradiation (400 nm) at a dose of 10 J/cm². Non-irradiated control condition (FIG. 15A) shows no appreciable cytotoxicity over control conditions, whereas irradiation with UV light under normoxic (FIG. 15C) and hypoxic (FIG. 15D) conditions demonstrated significant cytotoxicity (**p<0.01, ****p<0.0001). Comparison of toxicity in normoxic and hypoxic conditions demonstrates a decrease in efficacy of treatment under hypoxic until NP dose=2.5e11 particles/ml.

FIGS. 16A-K show results of alamar blue assay of PC3 cells treated with varying doses of radioPDT NP, control conditions of NSC encapsulated NP and PPIX, differing oxygenation conditions, and radiation doses (*p<0.05,**p<0.01,***p<0.001, ****p<0.0001).

FIGS. 17A-D show relative enhancement in cytotoxicity from radioPDT effect over the effect of radiation alone at 0Gy to 8Gy. The enhancement ratio is characterized in normoxic condition (20%) and in different degrees of hypoxia. Enhancement ratio was calculated by comparing cell viability assessed via alamar blue assay in RT only and RT+NP conditions.

FIG. 18 shows weight assessments of C57BL/6 mice injected with radioPDT NP in a serial dose escalation fashion. Mice were assessed for 48 hours before being sacrificed for post-mortem histopathologic analysis. A control group with PBS injection is included for comparison.

FIG. 19 shows post-mortem H&E analysis of select organs from the highest dose of IV-injected radioPDT NP group is shown on the left (1000 mg/kg). Confocal fluorescence microscopy (400 nm excitation, 639 nm emission) targeted at imaging the PPIX signal in the radioPDT NP shows their distribution in these organs (right).

FIGS. 20A-B show accumulation kinetics as quantified from CT imaging of the NP in the liver and tumor of IV (n−4) and IT (n=2) injected tumors.

FIG. 21A shows a MIP image of a mouse that demonstrated appreciable tumor accumulation (blue depicts H.U.>68). Light green is the outlined tumor, yellow is the outlined liver. Average H.U. values over time of the tumor are shown in FIG. 21B.

FIG. 22 shows a coronal view of a mouse with IT injected NP shown prior to injection, and 10 minutes, 1 hour, 4 hours, 24 hours and 48 hours post-injection. Orange circle depicts the tumor. Brighter areas indicate higher H.U. values and signal enhancement.

FIG. 23 shows tumor size measurements (via calipers) tracked between the 4 groups of the therapeutic study. For the values assessed so far, the RT+NP group achieved a significant decrease in tumor size over RT alone, as analyzed via two-tailed unpaired t-test (*p<0.05, **p<0.0097). Both RT only and RT+NP were strongly significantly different than the control groups of control and NP only (not shown). Control and NP only were not significantly different from each other.

### DETAILED DESCRIPTION OF PREFERRED EMBODIMENTS

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a,” “an,” and “the” include plural referents unless the context clearly dictates otherwise.

The present invention relates to a theranostic radiophotodynamic therapy nanoparticle, pharmaceutical compositions comprising same, and methods of preparing and using same. As used herein, the term “theranostic” refers to therapy which combines diagnostic and therapeutic capabilities in a single agent. As used herein, the term “radiophotodynamic therapy” (abbreviated as “radioPDT”) refers to radiation-activated photodynamic therapy. As used herein, the term “radiation” in the context of medicine refers to the use of controlled amounts of radiation for the treatment of diseases, particularly cancer. In one embodiment, radiation is conducted using X-rays. As used herein, the term “photodynamic therapy” refers to treatment which uses a photosensitizer which, upon exposure to a specific wavelength of light, produces a form of oxygen which kills cells. As used herein, the term “nanoparticle” refers to a particle having at least one dimension of 100 nanometres or less in size, and consequently has a high surface-to-volume ratio.

As will be described herein, the nanoparticle of the present invention comprises a nanoscintillator, a photosensitizer, and a nanocarrier. In one embodiment, the nanoparticle may optionally comprise one or more diagnostic agents, therapeutic agents, or a combination thereof. All components of the nanoparticle are further described. In addition, the invention relates to compositions comprising the nanoparticle, and methods of preparing and using the nanoparticles and compositions comprising same.

### Nanoscintillator

As used herein, the term “nanoscintillator” refers to a radioluminescent material which has at least one dimension of 100 nanometers or less in size. In one embodiment, the nanoscintillator ranges from about 10 nanometers to about 30 nanometers in length. The nanoscintillator may be selected from various organic and inorganic radioluminescent materials. The nanoscintillator may be doped by a dopant. As used herein, the term “dopant” refers to a substance which has been introduced into another substance. Suitable dopants include, but are not limited to, elements such as terbinium, yttrium, sodium, cerium, and the like. In one embodiment, the percentage of dopant ranges from about 0% to about 50%. In one embodiment, the nanoscintillator comprises lanthanum fluoride doped with 10% cerium. In one embodiment, the nanoscintillator is hydrophobic to enable its encapsulation within a nanocarrier. As used herein, the term “hydrophobic” means a physical property of repelling water.

The nanoscintillator may be operably associated with a photosensitizer by being either physically proximate to the photosensitizer via encapsulation within the nanocarrier, or physically linked or bonded to the photosensitizer. As used herein, the term “radioluminescent” refers to the ability to produce light by bombardment with ionizing radiation such as, for example, X-rays. The nanoscintillator exhibits particular desired wavelengths of radioluminescence in order to transfer energy to the photosensitizer via fluorescent resonant energy transfer (“FRET”). As used herein, the term “FRET” refers to a physical phenomenon whereby energy created by fluorescence exitation of the nanoscintillator is transferred to the photosensitizer.

In one embodiment, the nanoscintillator exhibits high attenuation of kilovolt range X-ray energies in order to function as a computed tomography (CT) contrast agent.

In one embodiment, the nanoscintillator exhibits a magnetic moment that affects the orientation of protons surrounding it in a magnetic field in order to function as a magnetic resonance imaging (MM) contrast agent.

### Photosensitizer

As used herein, the term “photosensitizer” refers to a molecule which absorbs light and produces a chemical change in another molecule in a photochemical process. Suitable photosensitizers include, but are not limited to, thiocyanates, vertiporfin, hypocrellin A, hypocrellin B, protoporphyrin IX, and the like. In one embodiment, the photosensitizer comprises protoporphyrin IX. In one embodiment, the photosensitizer is hydrophobic, enabling its encapsulation within the hydrophobic core of the nanocarrier.

The photosensitizer exhibits an absorption spectrum which overlaps with the luminescence spectrum of the nanoscintillator in order to absorb the luminescence of the nanoscintillator, thereby achieving adequate FRET efficiency.

### Nanocarrier

As used herein, the term “nanocarrier” refers to a material having at least one dimension of 100 nanometers or less in size for use as a transport module for the nanoscintillator, photosensitizer, and optionally one or more diagnostic agents, therapeutic agents, or a combination thereof. In one embodiment, the nanocarrier comprises a hydrophobic compound and a hydrophilic compound.

As used herein, the term “hydrophobic” means a physical property of repelling water. In one embodiment, the hydrophobic compound comprises a 7000 Da chain of poly lactide co-glycolide acid (PLGA). In one embodiment, the size of the hydrophobic PLGA compound ranges from about 3000 Da to about 30000 Da.

As used herein, the term “hydrophilic” means having an affinity for water. In one embodiment, the hydrophilic compound comprises a di-block co-polymer with a 5000 Da chain of methyl terminated polyethylene glycol (PEG). As used herein, the term “block copolymer” refers to a copolymer formed by two monomers clustering together to form blocks of repeating units. In one embodiment, the size of the hydrophilic PEG compound ranges from about 1500 Da to about 15000 Da.

In one embodiment, the hydrophilic compound may be functionalized by using a variant of PEG that has a termination other than methyl. For example, an amine group terminated PEG may be used to functionalize the nanoparticle with a targeting antibody including, but not limited to, prostate-specific membrane antibody to target prostate cancer, and herceptin antibody to target Her-2 over-expressing breast cancer. In this manner, the nanoparticle may be functionalized to actively target cancerous cells.

### Preparation of the Nanoparticles

In one embodiment, the process for preparing the nanoparticle comprises initially forming the “payload” and the nanocarrier as separate components, and then mixing the payload with the nanocarrier to form the resultant nanoparticle. The “payload” comprises the nanoscintillator, photosensitizer, and optionally, one or more diagnostic agents, therapeutic agents, or a combination thereof. The nanoscintillator may be separate from the photosensitizer, or linked or bound to the photosensitizer. The nanocarrier is intially prepared by linking or binding the hydrophobic compound with the hydrophilic compound.

The payload and the nanocarrier are mixed together in an aqueous solution, and aggregate to form the nanoparticle encapsulating the payload. The nanoparticle exhibits a micellar-like structure, including a hydrophobic core (i.e., the hydrophobic compound loaded with the nanoscintillator, photosensitizer, and optionally, one or more diagnostic agents, therapeutic agents, or a combination thereof); and a hydrophilic coating (i.e., the hydrophilic compound) surrounding the hydrophobic core and exposed to the aqueous environment. Without being bound by any theory, the hydrophilic coating allows for easier extravasation from tumor vasculature, and decreases opsinization, phagocytosis by macrophages, and clearance through the reticuloendothelial system.

In one embodiment, the nanoparticle comprising the nanocarrier, nanoscintillator, photosensitizer, and optionally, one or more diagnostic agents, therapeutic agents, or a combination thereof, has a size ranging from about 30 nm to 150 nm. In one embodiment, the size ranges from about 100 nm to about 120 nm. Without being bound by any theory, the nanoparticles may accumulate at a tumor through enhanced permeability and retention effect due to their size and presence of the hydrophilic compound. As used herein, the term “enhanced permeability and retention effect” refers to the tendency of nanoparticles of specific size to accumulate within tumors rather than normal tissues by taking advantage of the leaky endothelial layer of tumor vasculature and poor lymphatic drainage.

In one embodiment, the nanoparticle exhibits a polydispersity index less than 0.2. As used herein, the term “polydispersity index” refers to a measure of the distribution of molecular mass in a given polymer sample.

In one embodiment, the nanoparticle exhibits an encapsulation efficiency of the photosensitizer ranging from about 85% to about 92%. As used herein, the term “encapsulation efficiency” refers to the percentage of photosensitizer which is successfully entrapped into the nanoparticle.

The prepared nanoparticles may be evaluated by testing in various ways including, but not limited to, dynamic light scattering, zeta-potential measuring, transmission electron microscopy, electron microscopy based element mapping, UV-spectroscopy, stability assays, element mapping studies, radiofluorescence studies with a clinical orthovoltage irradiator, CT imaging, MRI imaging, IV administration in ex-ovo chicken chorioallantoic membrane models for plain-film X-ray imaging of vasculature and accumulation within tumors, assessment of singlet oxygen yield in normoxic and hypoxic conditions, and the like.

### Pharmaceutical Compositions Comprising the Nanoparticles

In another aspect, the invention comprises pharmaceutical compositions comprising the above nanoparticle in combination with one or more pharmaceutically acceptable carriers. As used herein, the term “carrier” means a suitable vehicle which is biocompatible and pharmaceutically acceptable, including for instance, liquid diluents which are suitable for administration. Those skilled in the art are familiar with any pharmaceutically acceptable carrier that would be useful in this regard, and therefore the procedure for making pharmaceutical compositions in accordance with the invention will not be discussed in detail. As used herein, the term “pharmaceutically acceptable” means a substance which does not significantly interfere with the effectiveness of the nanoparticle, and which has an acceptable toxic profile for the host to which it is administered. Suitably, the pharmaceutical compositions may be in the form of liquids and solutions suitable for injection in liquid dosage forms as appropriate and in unit dosage forms suitable for easy administration of fixed dosages. The dosage of the nanoparticle depends upon many factors that are well known to those skilled in the art, for example, the type and pharmacodynamic characteristics of the nanoparticle; age, weight and general health condition of the subject; nature and extent of symptoms; any concurrent therapeutic treatments; frequency of treatment and the effect desired.

### Uses of the Nanoparticles and Compositions Comprising Same

The nanoparticles and compositions comprising same may be used in various applications including, but not limited to, medical, veterinary, and dental. Exemplary nanoparticles of this invention are biocompatible and intended for medical applications. As used herein, the term “biocompatible” means generating no significant undesirable host response for the intended utility. Most preferably, biocompatible compositions are non-toxic for the intended utility. Thus, for human utility, biocompatible is most preferably non-toxic to humans or human tissues. The nanoparticles are selective for tumors and non-toxic to healthy cells.

Certain embodiments of the invention thus relate to methods and uses of the nanoparticles. In one embodiment, the invention comprises a method of treating, preventing, or ameliorating a disease in a subject, comprising administering to the subject an effective amount of the above nanoparticle, or a pharmaceutical composition comprising same; and applying radiation to the subject. In one embodiment, the nanoparticle or pharmaceutical composition is administered intravenously. In one embodiment, the radiation is conducted using X-rays. In one embodiment, the invention comprises use of the above nanoparticle, or a pharmaceutical composition comprising same to treat, prevent, or ameliorate a disease in a subject.

As used herein, the term “disease” includes, but is not limited to, cancer, dermatological conditions, infections, macular degeneration in the eye, Barrett's esophagus, deep abdominal abscesses, or any condition in which specific and directly controlled cell cytotoxicity is desired. In one embodiment, the disease is selected from prostate cancer or breast cancer. As used herein, the term “subject” means a human or other vertebrate. As used herein, the term “effective amount” means any amount of a formulation of the nanoparticle useful for treating, preventing, or ameliorating a disease upon administration. An effective amount of the composition provides either subjective relief of symptoms or an objectively identifiable improvement as noted by the clinician or other qualified observer. As used herein, the terms “treating,” “preventing” and “ameliorating” refer to interventions performed with the intention of alleviating the symptoms associated with, preventing the development of, or altering the pathology of a disease, disorder or condition. Thus, in various embodiments, the terms may include the prevention (prophylaxis), moderation, reduction, or curing of a disease, disorder or condition at various stages. In various embodiments, therefore, those in need of therapy/treatment may include those already having the disease, disorder or condition and/or those prone to, or at risk of developing, the disease, disorder or condition and/or those in whom the disease, disorder or condition is to be prevented.

In one embodiment, the nanoparticle is theranostic, and capable of performing both diagnostic imaging and photodynamic therapy using radiation, particularly X-rays, as its activating source. External radiation may be used to irradiate the nanoparticle precisely at the target such as, for example, a tumor. The radiation activates the nanoscintillator which luminesces or emits particular desired wavelengths of radioluminescence in order to transfer energy to the photosensitizer. The photosensitizer exhibits an absorption spectrum which overlaps with the luminescence spectrum of the nanoscintillator in order to absorb the luminescence of the nanoscintillator, thereby achieving adequate FRET efficiency. The photosensitizer produces a form of oxygen (for example, singlet oxygen species) which kills the target such as, for example, a tumor.

The nanoparticles thus combine the strengths of radiotherapy with those of photodynamic therapy to address the limitations of both treatment modalities. The use of photodynamic therapy to augment radiotherapy means the therapeutic effect to the tumor can be boosted without adding any extra dose of radiation and minimizing radiation toxicity, which ultimately increases the overall therapeutic ratio. The use of X-rays to activate the nanoparticles is advantageous because X-rays can penetrate deep into tissues, and modern radiotherapy has very precise methods of delivering X-rays to any location within the body. This effectively overcomes the limitation of photodynamic therapy which uses visible spectrum light for therapeutic activation, and thus cannot reach deep-seated tumors.

The imaging enhancing capability and ability of the nanoparticle to localize in a tumor when encapsulated in a nanocarrier may be used for radiation targeting in image guided radiotherapy (IGRT) via pre- or post-treatment CT scanning, or real-time imaging with MRI. The imaging enhancing capability may also be used for treatment delivery quantification of radiophotodynamic therapy via extrapolation of the amount of the nanoparticle within the target based on the CT or Mill signal, and the activating radiation dose delivered to the target.

In one embodiment, the nanoparticle may comprise one or more therapeutic agents. In one embodiment, the therapeutic agent comprises a cytotoxic drug. The therapeutic agent may be encapsulated within the hydrophobic core of the nanoparticle, preventing its release and suppressing its activity or effect while in transport. Once the nanoparticles are positioned within the target area, they may be activated by radiation, initiating radiophotodynamic therapy and producing singlet oxygen species. Without being bound by any theory, the singlet oxygen species not only exert cytotoxic effects, but also destroy the nanocarrier, thereby degrading the nanoparticle. Breakdown of the nanoparticle in turn releases the encapsulated therapeutic agent locally at the target area. In this manner, targeted delivery of the therapeutic agent may be achieved with precision in release and distribution. As an example, this bestows a concurrent mode of localized therapy for deep seated tumors at the same time.

It should be apparent, however, to those skilled in the art that many more modifications besides those already described are possible without departing from the inventive concepts herein. The inventive subject matter, therefore, is not to be restricted except in the scope of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

In the development of the invention, it is known that the radiotherapeutic effect on cancer tissues is dependent on environmental oxygen and oxyradical formation to fix potentially reversible DNA damage from ionizing radiation. PDT consumes environmental oxygen to generate the highly reactive singlet oxygen species to cause cellular and organelle damage. Through previous hypoxia studies, PDT's therapeutic effect is limited to half of normoxic maximal cell-kill rate (½ Kₘₐₓ) at 1% oxygenation, and for radiotherapy this ½ Kmax is at 0.5% oxygenation. Thus, one could suppose that as such hypoxic conditions are approached, there may be a competition for oxygen between these competing effects. However, PDT is known to rapidly consume the environmental oxygen, with most of the singlet oxygen species generated in less than a microsecond. Direct damage from radiotherapy relies on direct double-strand DNA breaks from ionizing radiation and is not oxygen dependent. Indirect radiotherapy damage on the other hand, partly depends on radiolysis generating hydroxyl radicals from water first that damage DNA structure, and then oxygen fixation of this DNA damage, with the process occurring in microseconds. Therefore, one could presume the PDT effect could rapidly convert the environmental oxygen into singlet oxygen to generate PDT effect, and either work in parallel or even synergistically with direct DNA damage from ionizing radiation. To date, there exists little data in conditions where these two effects occur simultaneously.

The inventors thus sought out to demonstrate that, under hypoxic conditions, one nanoscintillator, cerium doped lanthanum fluoride (Ce:LaF₃), and PPIX loaded PEG-PLGA nanoparticles will show significant radioPDT effect via clinically useful singlet oxygen production in combination with radiotherapy, leading to greater cytotoxicity to cancerous cells and better tumor control when compared to radiotherapy alone. The first aim was to synthesize novel radioPDT nanoparticle using the nanoscintillator Ce:LaF₃, the photosensitizer PPIX, and the nanocarrier PEG-PLGA. The second aim involved radioPDT singlet oxygen quantification in hypoxic conditions to characterize and quantify the singlet oxygen yield from radioPDT under differing oxygenation conditions, nanoparticle concentrations, and radiation doses. The third aim was to characterize baseline nanoparticle toxicity, radiation toxicity, and compare it to cytotoxicity of combined radiation with radioPDT in prostate cancer cell line PC3 in vitro and in vivo.

Based on preliminary studies described in the Examples, the inventors have found that synthesized nanoparticles of the present invention exhibit good reproducibility, size, and stability characteristics. Their characteristics in terms of toxicity in vitro and in vivo is very low. Functional studies have demonstrated its capability to produce contrast enhancement for imaging and radioPDT effect for therapy. Hypoxic conditions will not limit the added benefit of combining radioPDT with radiotherapy. In vitro studies show significant therapeutic effect over RT alone, and is a function of NP dose, RT dose and hypoxic condition. Therapeutic effect was still maintained in hypoxic condition and sufficient NP and RT doses. In vivo studies demonstrate its potential for diagnostic and therapeutic effects with prostate cancer cell lines.

Embodiments of the present invention are described in the following Examples, which are set forth to aid in the understanding of the invention, and should not be construed to limit in any way the scope of the invention as defined in the claims which follow thereafter.

### Example 1—Synthesis of radioPDT Capable Ce:LaF₃ and PPIX Encapsulated PEG-PLGA Nanospheres

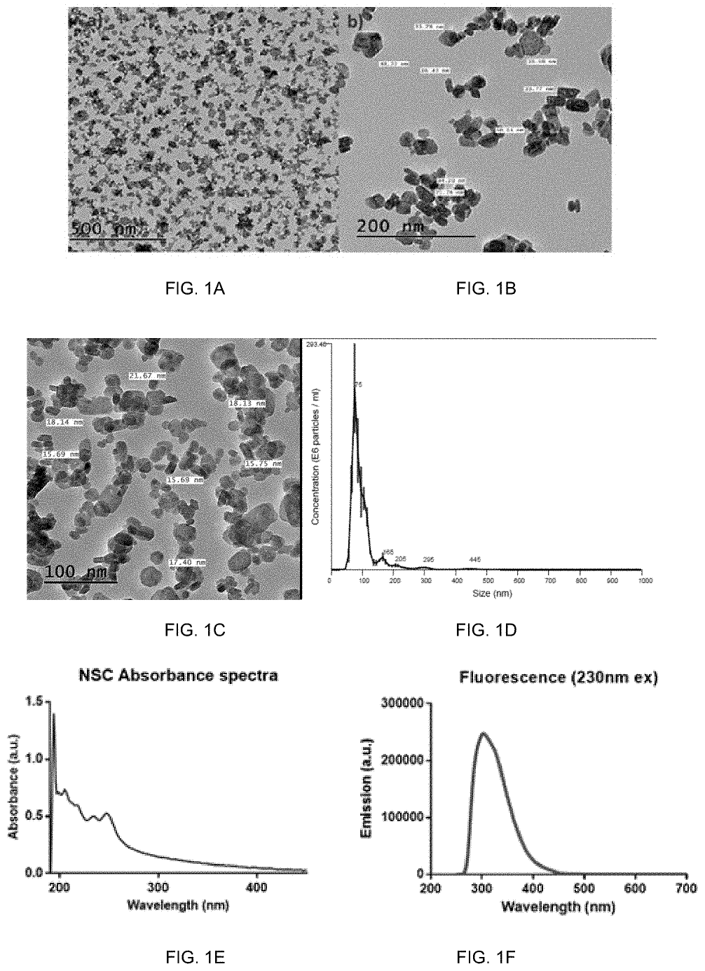

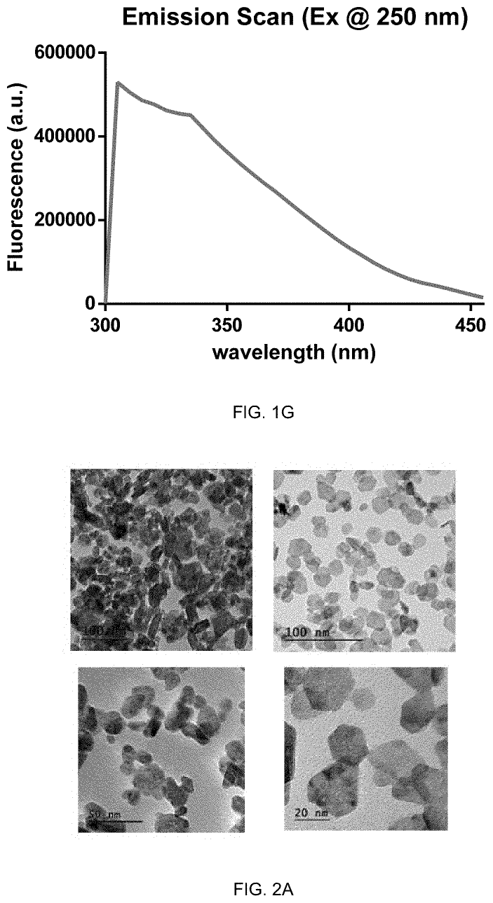

The nanoscintillators were produced using wet chemistry synthesis, with the temperature, solution, and concentration of reagents optimized to produce particles of the desired size. La[NO₃]₃ (9 mmol), Ce[NO₃]₃ (1 mmol), and NH₄F (30 mmol) were dissolved in water with stirring for about 30 minutes. The mixture was heated under nitrogen protection to 100° C. and stirred until a colloidal suspension formed. The mixture was centrifuged and washed with deionized water, followed by washing with acetic acid and re-dissolving in water. The nanoscintillators were characterized using TEM (FIGS. 1A-C). Quantification studies with DLS-based measurements demonstrated a particle count of (9±1)×10¹¹ particles per mL (FIG. 1D). ICP-MS showed a lanthanum concentration of 3016 ppm and cerium concentration of 392 ppm in the final solution. The UV-Vis absorbance spectroscopy revealed expected absorbance peaks for 10% cerium doped lanthanum fluoride (FIG. 1E). Emission spectra (FIG. 1F-G) showed emission peaks which correspond with expected values.

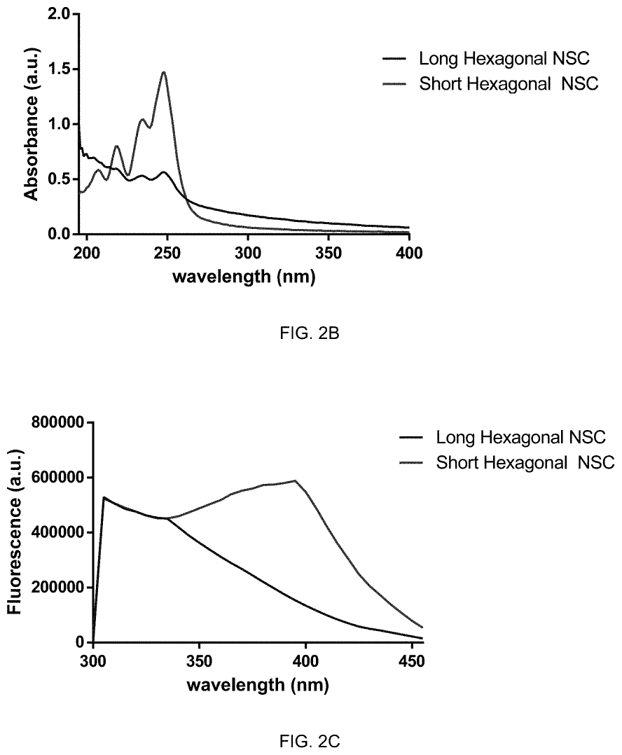

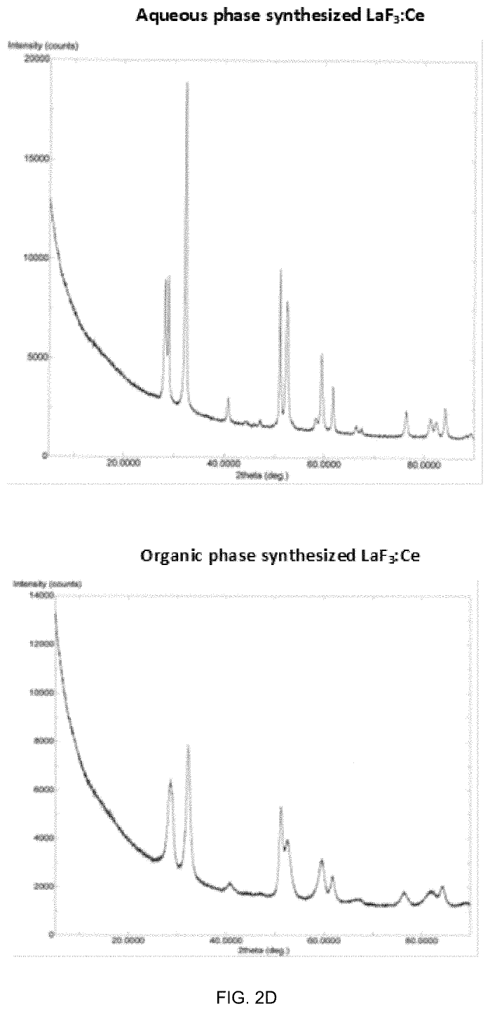

Switching from aqueous media to anhydrous methanol yielded more readily dispersible nanoparticles, and improved X-ray attenuation and fluorescence characteristics. FIGS. 2A-D show LaF₃:Ce nanoscintillators synthesized in aqueous vs. organic media under TEM (FIG. 2A). Differences in relative intensities of absorption peaks are demonstrated (FIG. 2B), along with fluorescence spectra (FIG. 2C). The XRD profiles of the aqueous phase and the organic phase synthesized LaF₃:Ce nanoscintillators are shown (FIG. 2D). XRD analysis shows a hexagonal crystal lattice structure.

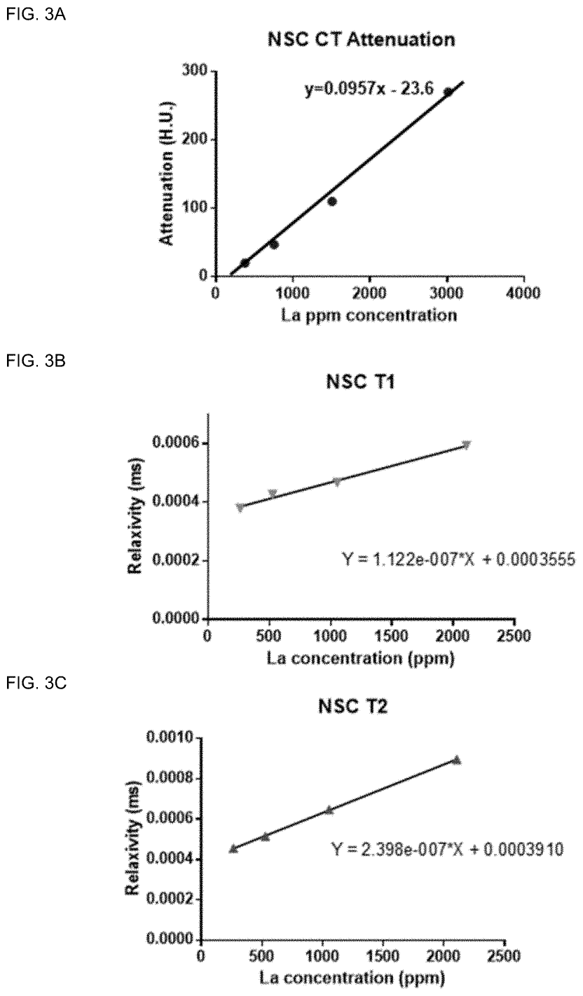

FIG. 3A is a graph showing nanoscintillators CT attenuation measured with a Varian TruBeam STx cone beam imager showing signal enhancement of 0.0957 Houndsfield(H.U.)/ppm. FIG. 3B is a graph showing MM signal inversion time analysis with a Philips 3 Tesla MRI measuring a T1 relaxivity constant of 1.122×10⁻⁷ ms/ppm. FIG. 3C is a graph showing MM signal inversion time analysis with a Philips 3 Tesla MRI measuring a T2 relaxivity constant of 2.398×10⁻⁷ ms/ppm.

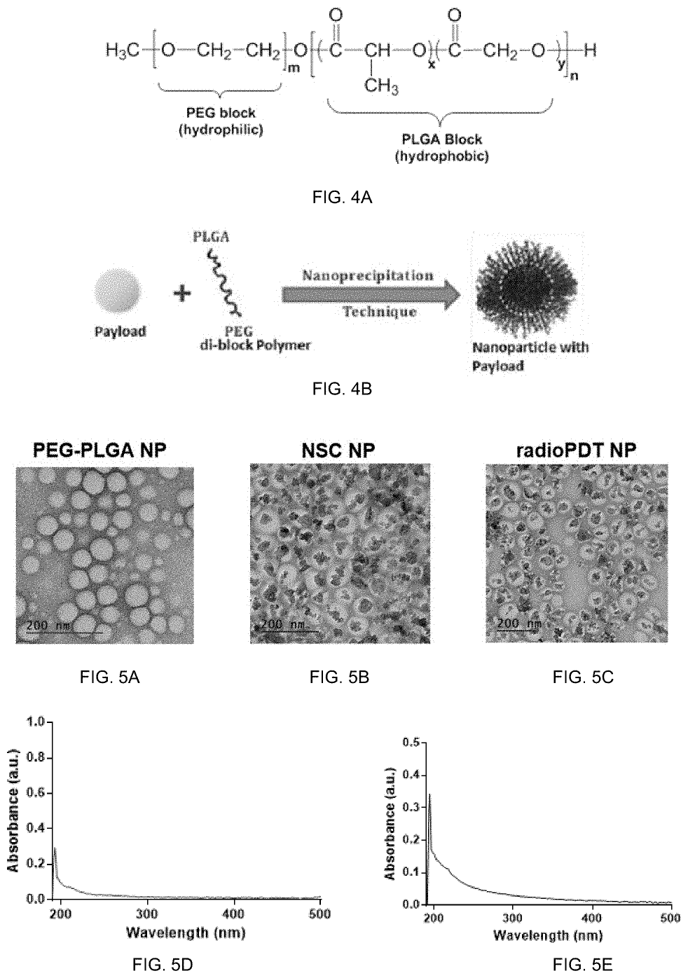

FIG. 4A is a schematic diagram showing the chemical structure of polyethylene glycol-polylactic acid-co-glycolic acid (PEG-PLGA). FIG. 4B is a schematic diagram showing a method of nanoparticle formation with payload encapsulation.

The nanoscintillators were encapsulated along with PPIX in PEG-PLGA nanosphere based nanoparticles using a single emulsion technique. The starting reagent concentrations, single emulsion drop rate, and reaction mixture stirring time were optimized to yield particles of about 100 nm. PEG-PLGA (5000/7000 Da) was dissolved in acetonitrile. PPIX and Ce:LaF₃ were added to an organic phase and dropped into water. The mixture was sonicated or stirred to disperse PEG-PLGA evenly. The mixture was vacuum-evaporated to remove the organic phase and centrifuged. The nanoparticles were washed with water.

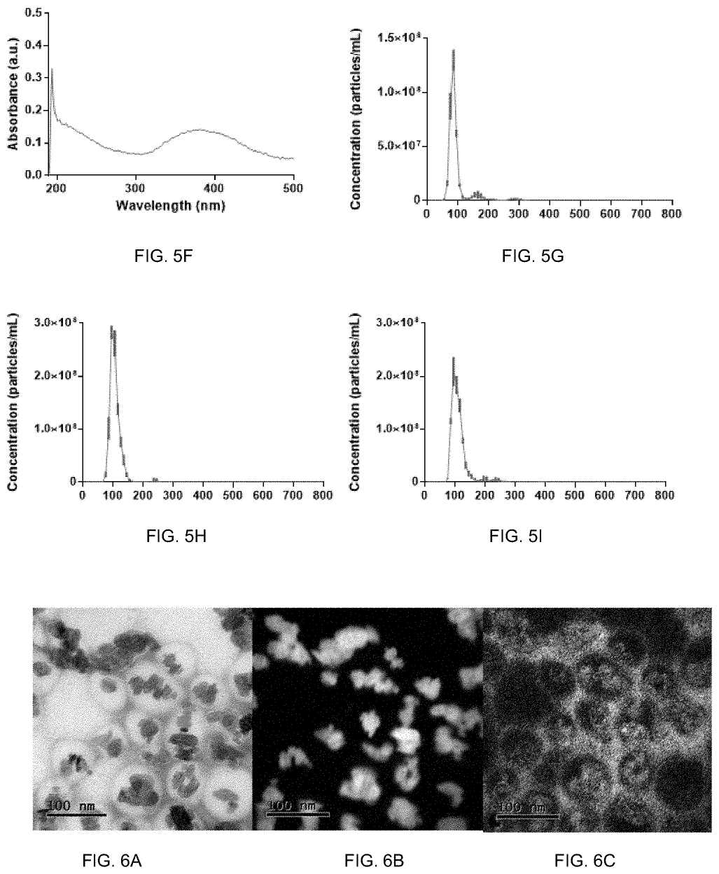

The nanoparticles (NP) were characterized using DLS, zeta potential, and TEM imaging. The size range was close to the desired 100 nm size on DLS and appeared similar in size when viewed with TEM (FIGS. 5A-C). The polydispersity index was below 0.1 by DLS. The zeta potential was zero to slightly negative as expected, given the methyl terminated PEG outer layer and carboxyl terminated PLGA core. The corresponding UV-Vis spectroscopy for each condition is shown in FIGS. 5D-F, and the concentration as a function of size via dynamic light scattering (DLS) is shown in FIGS. 5G-I. As measured by DLS, the mean size for each variant is 75, 95 and 125 nm, respectively, with a polydispersity index of <0.3.

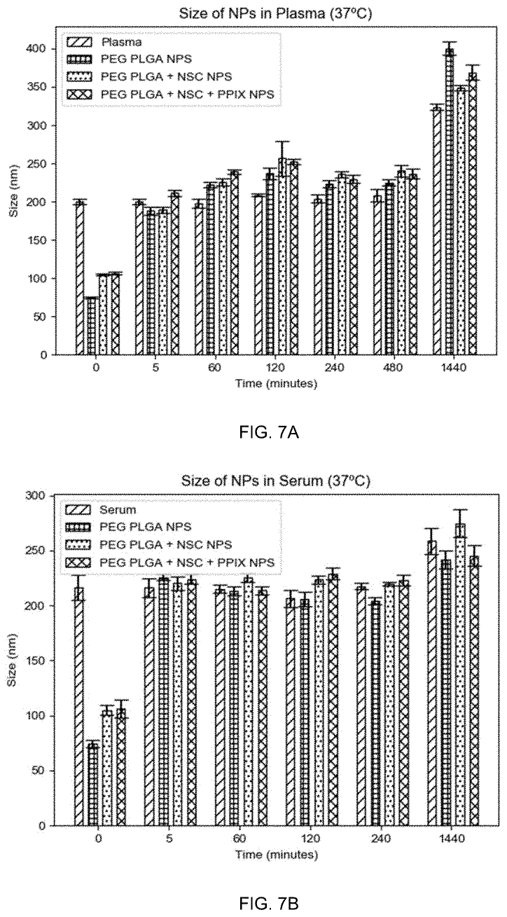

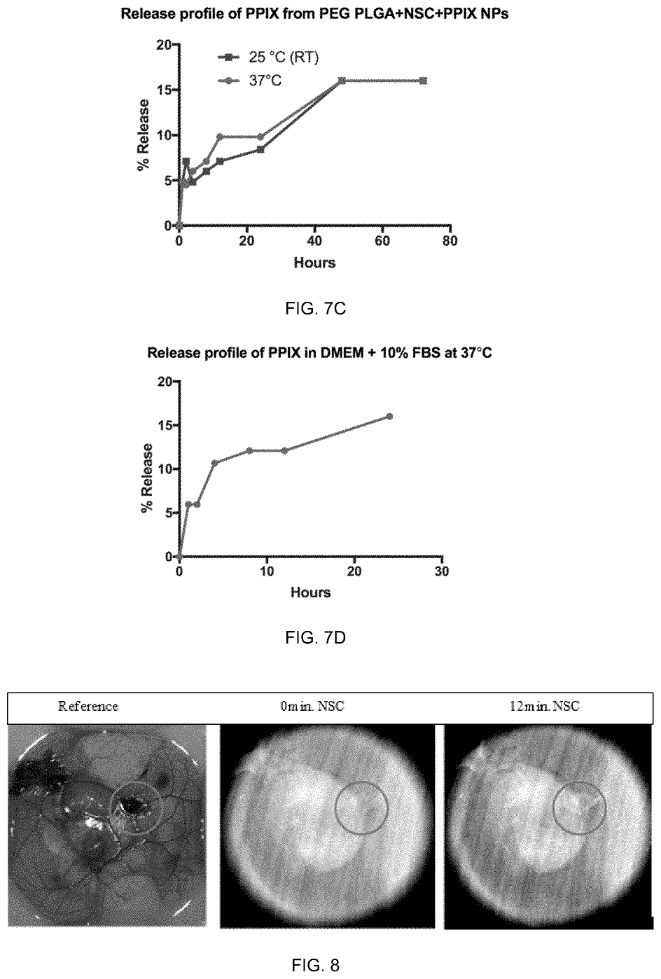

Encapsulation studies were conducted using UV-Vis spectroscopy and elemental mapping with electron energy loss TEM (FIGS. 5D-F, 6A-C). The UV-Vis data showed the peaks of the nanoscintillator, and the broad 400 nm peak of PPIX in the appropriate nanoparticles. The encapsulation efficiency of PPIX was calculated to be 92% on average between multiple synthesis batches. The elemental mapping images showed the lanthanum element signal of the nanoscintillator to be within the oxygen signal of the PEG-PLGA, suggesting that the nanoscintillators were being encapsulated successfully within the microsphere. Release characteristics of the PPIX over time were measured by incubating the PPIX and nanoscintillator encapsulated nanoparticle in phosphate buffered saline media (pH=7.4). The nanoparticles exhibited high stability and slow release over 48 hours at room (24° C.) and body (37° C.) temperatures (FIG. 7A). The size of the nanoparticles remained stable for the first 24 hours, and gradually increased over the next 24 hours (FIGS. 7B-D), suggesting aggregation of the nanoparticles with prolonged exposure to these conditions. FIG. 8 shows X-ray images of a chick chorioallantoic membrane (CAM) model with HT1080 tumors implanted on day 10 with X-ray imaging studies done on day 15 and 16. The CAM was sequentially imaged immediately after IV injection of NSC and 12 min later to demonstrate intra-tumoral accumulation over time (circle).

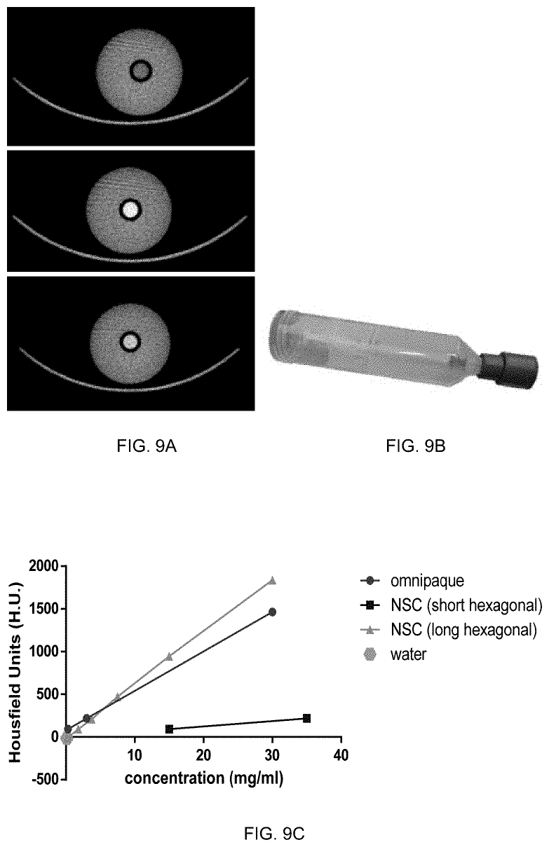

A peristaltic pump and tangential flow filtration (TFF) machine replaced the hand-controlled drop-wise nanoprecipitation and centrifugation-based purification, allowing the synthesis of nanoparticles to be scaled up approximately 100× to allow up to 500 mg per batch to be synthesized. The nanoparticles were assessed for diagnostic capabilities via CT attenuation characteristics. Using the Siemens preclinical microCT, the NSC were imaged in a mouse phantom (FIGS. 9A-B). The signal from the NSC solution was quantified for Hounsfield units and plotted against the control condition of omnipaque CT contrast dye that was imaged under the same conditions. Both the long hexagonal NSC and short hexagonal NSC were imaged to generate an attenuation-dose curve. Both structures of NSC demonstrated appreciable signal gain, but the short hexagonal NSC had a much better performance in comparison to omnipaque (FIG. 9C).

### Example 2—Singlet Oxygen Studies Yield Demonstrate Significant Singlet Oxygen at Normoxic and Hypoxic Conditions

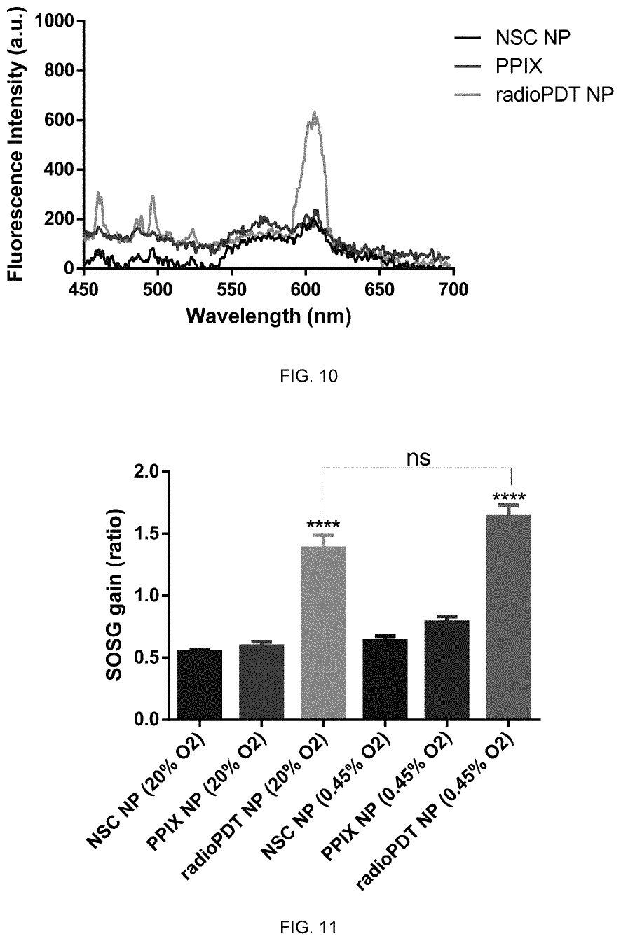

The synthesized NPs were tested for functionality in performing radioPDT. FRET activity was assessed using fluorescence spectroscopic analysis (FIG. 10). An excitation wavelength of 200 nm was used, which is within the absorption spectrum of the NSC, but outside the excitation of PPIX. The ability of the radioPDT NP to absorb the excitation wavelength and transfer energy to the PPIX via FRET was demonstrated by the rise in light emission at the maxima of the PPIX, which was not seen in control conditions that precluded FRET.

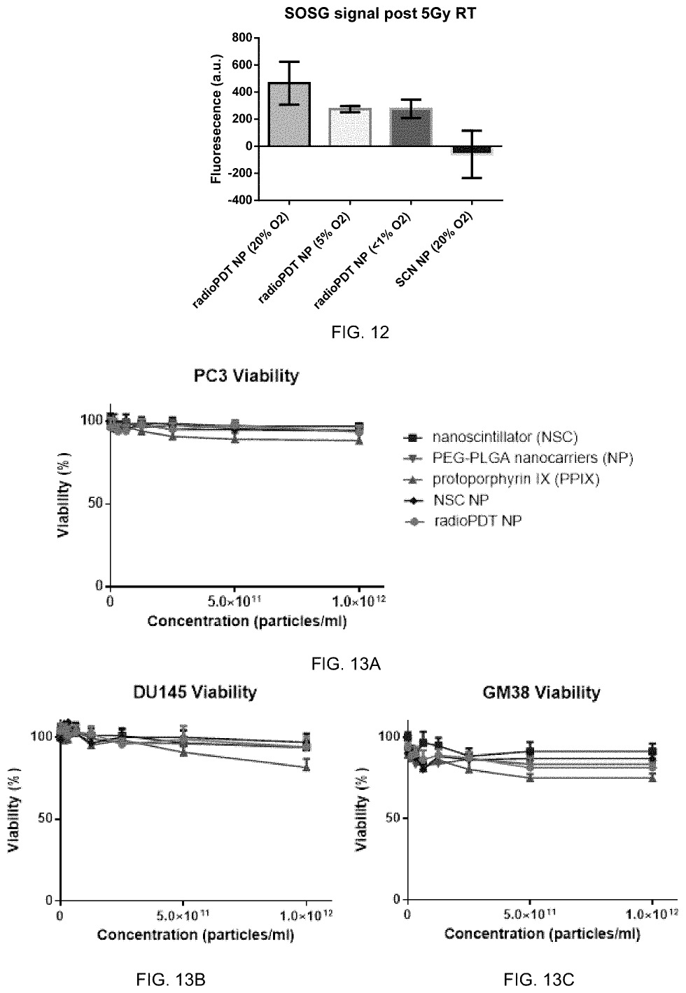

Photodynamic functionality was assessed using singlet oxygen yield via a commercially available fluorescent probe Singlet Oxygen Sensor Green (SOSG) specific for singlet oxygen over other ROS species (FIGS. 11 and 12). Under X-ray radiation with a 300 kV linear accelerator, an appreciable singlet oxygen signal was detected above control conditions (FIG. 11). This effect was observed both in normoxic conditions and hypoxic conditions down to 0.45% and RT doses of 5 Gy and greater. With NP concentrations greater than Sell particles/ml, a similar signal gain from the SOSG probe can be demonstrated in both normoxic and hypoxic conditions.

### Example 3—In Vitro Studies Demonstrate Low In Vitro Toxicity, Cellular Uptake in Prostate Cancer Cells, and High Therapeutic Effect when Activated with Radiation

In an inactivated form (outside the field of radiation), the nanoparticles are expected to be relatively nontoxic. To confirm, baseline toxicity experiments were conducted with the fully assembled nanoparticles and its constituents. The NSC, PPIX, PEG-PLGA only NP, NSC encapsulated NP, and NSC with PPIX encapsulated NP were tested for cytotoxicity against normal skin fibroblast cell line GM38, and prostate cancer cell lines of PC3 and DU145. MTT assays detected changes in cell proliferation and viability. Alamar blue proliferation assay was used to determine viability, along with light microscopy to confirm cell death (not shown). No appreciable differences were seen over increasing doses of the NP or their substituents (FIGS. 13A-C), suggesting the nanoparticles in the inactive form are relatively nontoxic.

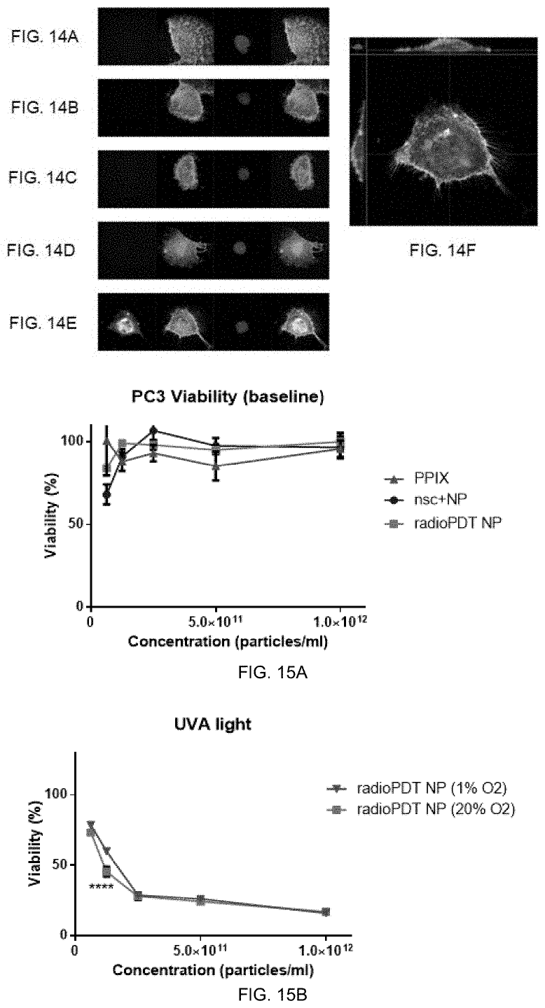

Further in vitro studies via confocal microscopy showed that the NPs can be taken up by PC3 prostate cancer cells into the cytoplasm, possibly by micropinocytosis (FIGS. 14A-F).

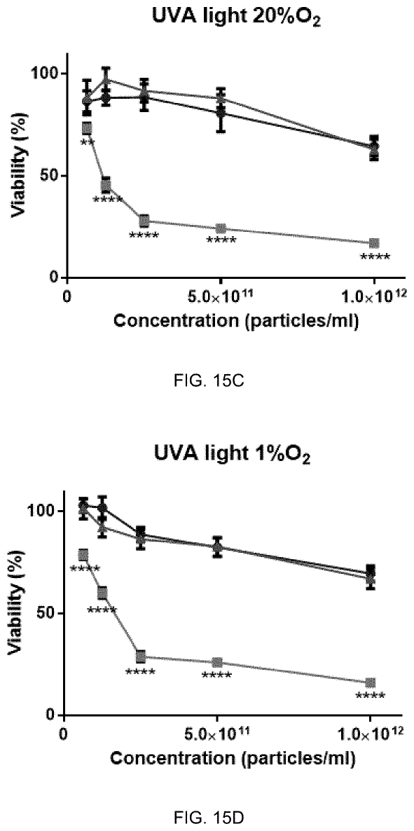

Therapeutic efficacy of the radioPDT NP was assessed using Alamar blue assay with PC3 cell line. Its effect under light PDT was confirmed using a 400 nm excitation source (FIGS. 15A-D). Significant cytotoxicity was observed in normoxic and hypoxic conditions, although until a threshold dose of NP was achieved, hypoxia appeared to decrease efficacy of the treatment significantly (FIG. 15B).

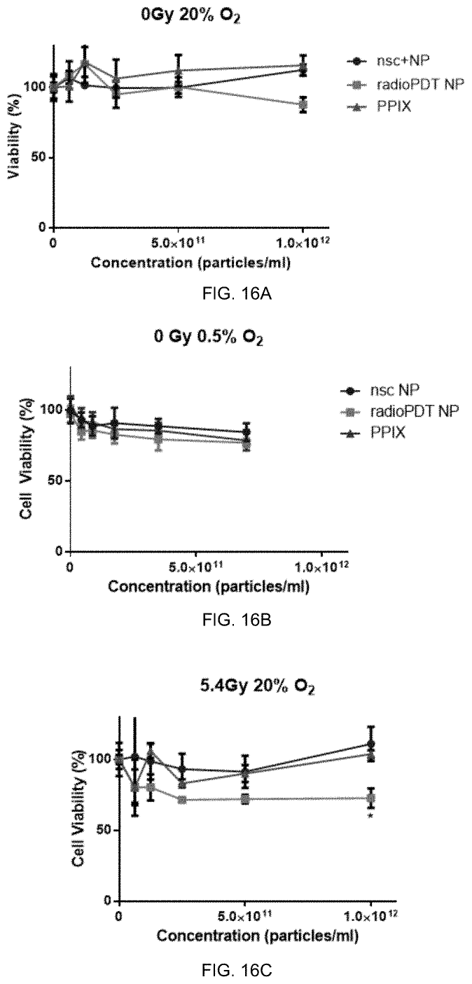

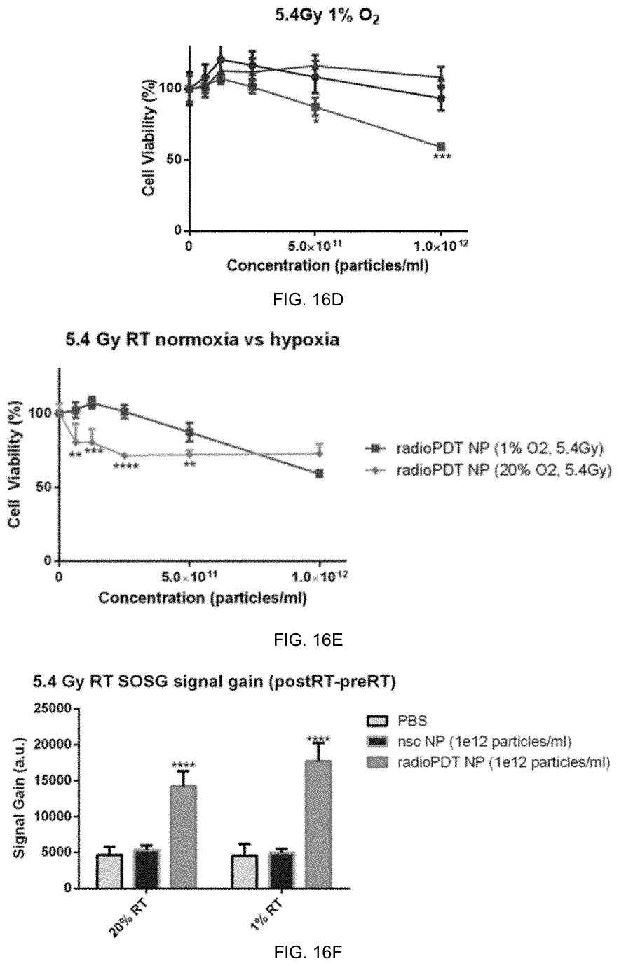

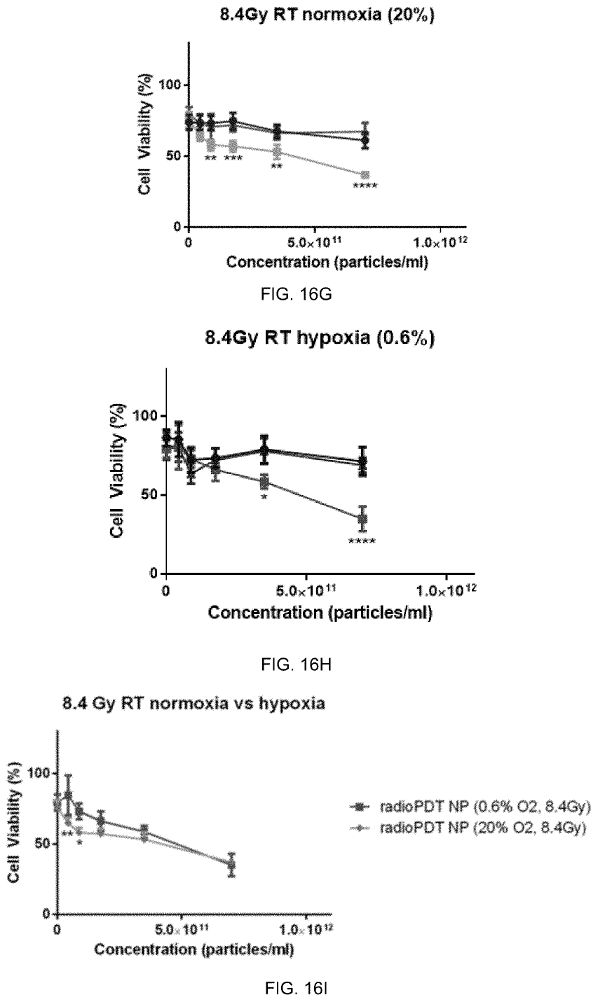

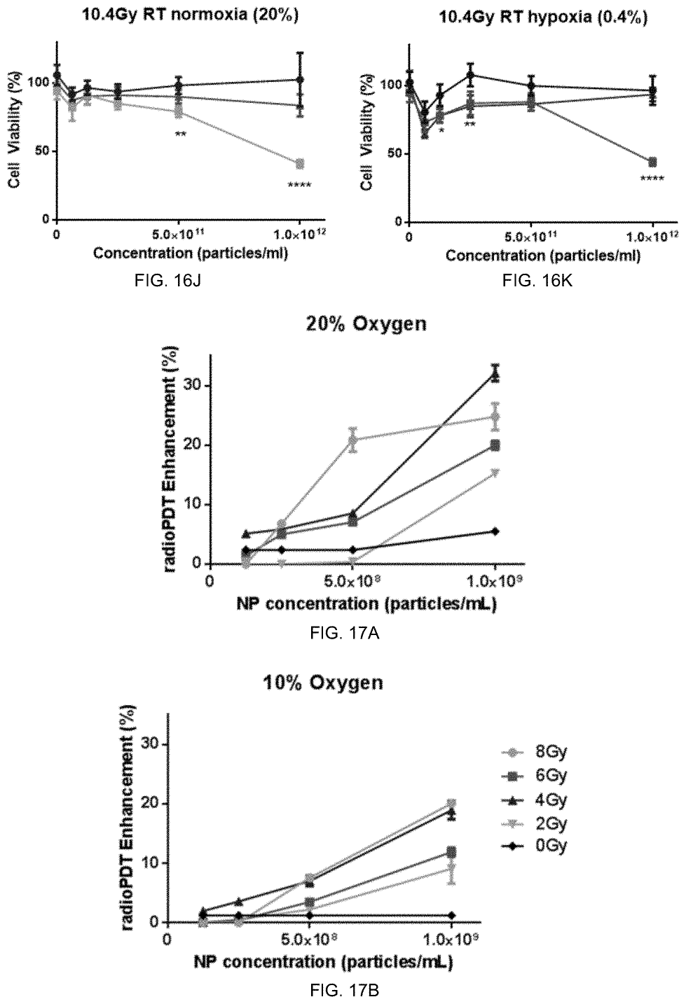

Therapeutic efficacy over baseline conditions was also observed with radiotherapy (FIGS. 16A-K). This was a dose dependent effect by RT dose and radioPDT NP dose. A threshold value of NP dose may be crossed before normoxic and hypoxic conditions demonstrate similar levels of cytotoxicity.

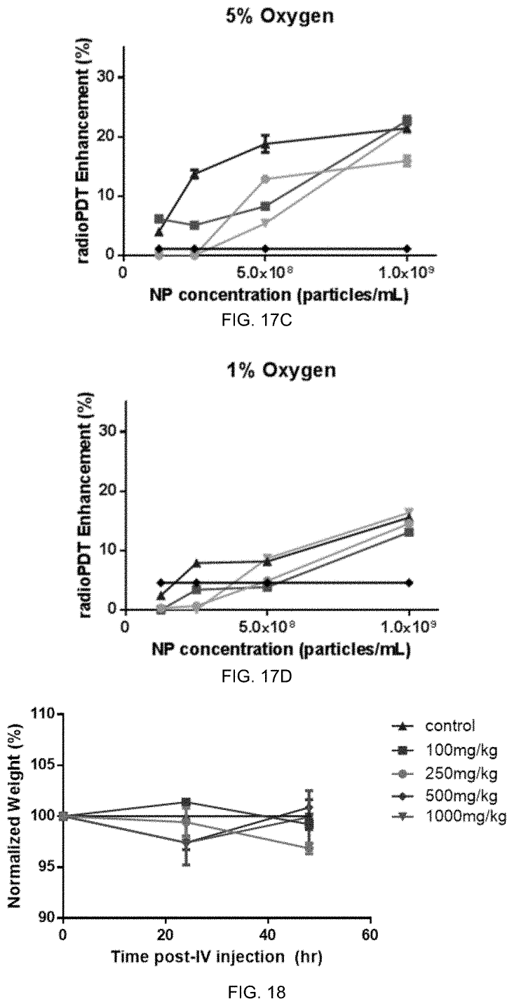

The cytotoxicity conferred by radioPDT under gradients of radiation dose, NP dose, and oxygenation conditions was examined (FIGS. 17A-D). The NP dose, oxygen concentration, and radiation dose is generally proportional to the enhancement ratio, ranging from up to 30% in normoxia, to 15% in hypoxia. The type of relationship between NP dose and RT dose appeared to be variable depending on the oxygenation and RT dose.

### Example 4—In Vivo Characterization of Toxicity, Diagnostic Potential, and Therapeutic Effect

The radioPDT NP's performance in vivo was assessed. In an acute toxicity study in C57BL/6 mice, dose escalation studies were started at approximately 100 mg/kg based on literature mice data on dosing PEG-PLGA nanospheres, with a target dose of 300 mg/kg to 500 mg/kg to be reached. The animals were injected via tail vein injections. Toxicity was assessed by behavioral changes in activity, fur changes, feeding, and weight changes over a 48 hour period. After 48 hours, the animals were sacrificed, and the heart, lung, liver, spleen, and kidneys harvested to for histopathologic analysis. The dose was administered to 2 mice per group and was escalated until dose-limiting toxicity of 10% weight loss or significant behavioral changes were reached.

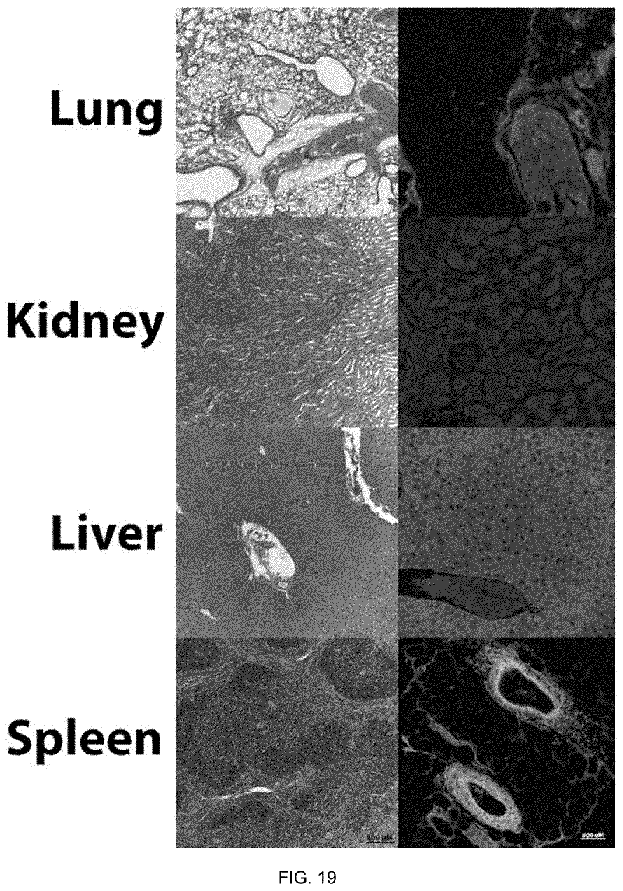

The acute toxicity experiment progressed until a dose of 1000 mg/kg was reached, as the NP were unstable in suspension at higher doses. No behavioral toxicity or weight loss was noted among any of the animals (FIG. 18). Histopathologic analysis of gross and microscopy histology revealed no signs of organ toxicity. In mice injected with more than 500 mg/kg, pigmentation of the spleen was noted, but no inflammation, necrosis, or organ damage was noted on H&E stain (FIG. 19). Confocal fluorescence microscopy targeted at imaging the PPIX (400 nm excitation, 639 nm emission) revealed the PPIX in the radioPDT NP was visible mostly in the spleen, liver, and pulmonary vasculature (FIG. 18) which is consistent with clearance by the reticuloendothelial system.

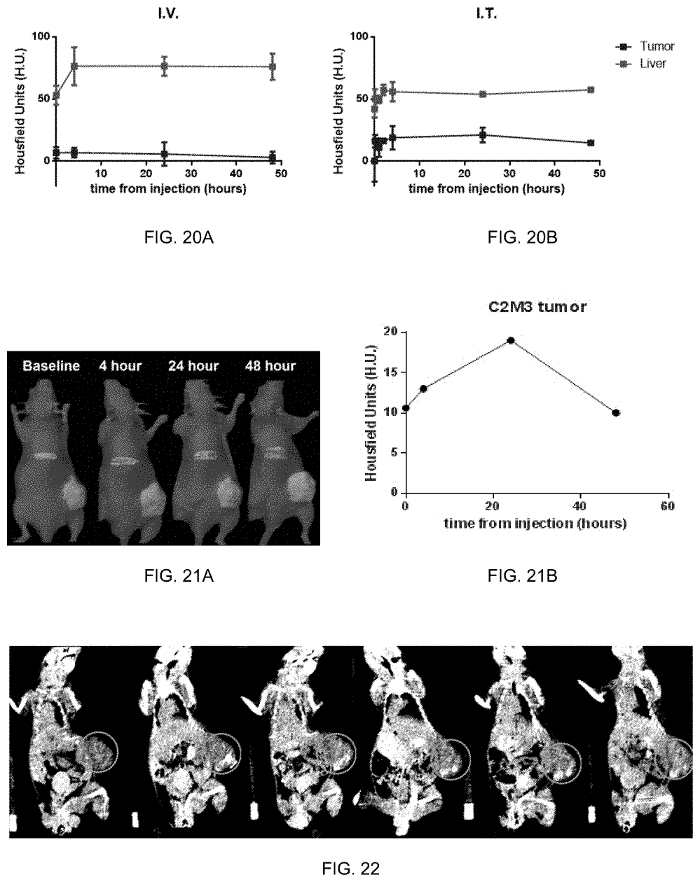

In vivo diagnostic characteristics of the radioPDT NP were assessed using the preclinical Siemens microCT scanner along with subcutaneous PC3 implanted NOD-SCID-gamma (NSG) mice models. Tumors were grown to 1000 mm³ before the mice were injected IV (tail vein) with 500 mg/kg of NP. Serial images were acquired at pre-injection baseline and post-injection at 4 hours, 24 hours, and 48 hours. A second cohort of mice were imaged with intratumoral (I.T.) injection as a comparator and imaged at baseline pre-injection and post-injection at 10 minutes, 1 hour, 4 hours, 8 hours, 24 hours and 48 hours. The images were analyzed using eclipse RT planning software for Hounsfield units in the liver and tumor (FIGS. 20A-B). The quantification of the contrast enhancement visible on CT demonstrated clear uptake in the liver, but no systematic trend in uptake in the tumor when injected IV (FIG. 20A). With I.T. injection, the uptake in the tumor was clear, and clearance into the liver, signified by increase in signal over time, was also noted (FIG. 20B). Some of the IV injected mice did show an appreciable gain in signal in the tumor (FIGS. 21A-B). The accumulation of NP in the IT injected tumors was clearly visible on CT as well (FIG. 22).

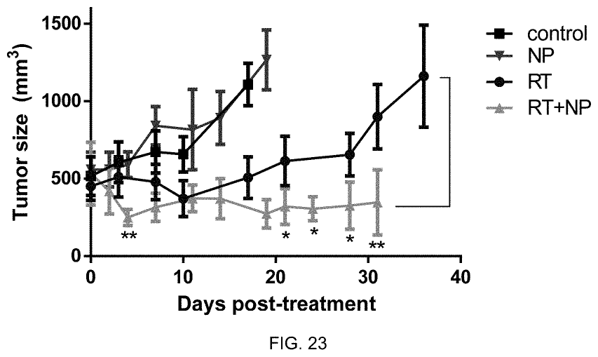

Therapeutic studies in vivo were conducted using another cohort of PC3 flank tumor-implanted NSG mice, with starting tumor size of 300-500 mm³. Four study groups of mice (n=4 per group) were used to evaluate treatment efficacy of the NP, and were divided into control, RT only, NP only, and RT along with NP (RT+NP) groups. The radioPDT NP (or PBS for the control group) was injected IT 24 hours prior to RT, which was conducted with the SAARP with a single fraction of 6 Gy to the tumor. Tumor response was evaluated via caliper-based tumor size measurements over time until endpoints of tumor size, wet ulceration, or poor animal health was reached. Tumor size measurements demonstrated no difference in tumor growth or survival in the control and NP only groups, and significantly slower growth characteristic of the RT+NP as compared to the RT only group (FIG. 23).

### REFERENCES

All publications mentioned are incorporated herein by reference (where permitted) to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.
Azzouzi, A.-R., et al., Padeliporfin vascular-targeted photodynamic therapy versus active surveillance in men with low-risk prostate cancer (CLIN1001 PCM301): an open-label, phase 3, randomised controlled trial. Lancet Oncology, 2017. 18(2): p. 181-191.
Chen, W. and J. Zhang, Using nanoparticles to enable simultaneous radiation and photodynamic therapies for cancer treatment. J Nanosci Nanotechnol, 2006. 6(4): p. 1159-66.
Cheng, J., et al., Formulation of functionalized PLGA-PEG nanoparticles for in vivo targeted drug delivery. Biomaterials, 2007. 28(5): p. 869-76.
Clement S., Deng W., Camilleri E., Wilson B. C. and Goldys E. M. (2016). X-ray induced singlet oxygen generation by nanoparticle-photosensitizer conjugates for photodynamic therapy: determination of singlet oxygen quantum yield. Scientific Reports. 6:19954.
Debele, T. A., S. Peng, and H. C. Tsai. (2015) Drug Carrier for Photodynamic Cancer Therapy. Int J Mol Sci 16(9): p. 22094-136.
Ding, H. et al., Nanoscopic micelle delivery improves the photophysical properties and efficacy of photodynamic therapy of protoporphyrin IX. J Control Release, 2011. 151(3): p. 271-7.
Elmenoufy, A. H. et al., A novel deep photodynamic therapy modality combined with CT imaging established via X-ray stimulated silica-modified lanthanide scintillating nanoparticles. Chem Commun (Camb), 2015. 51(61): p. 12247-50.
Fang J., Liao L., Yin H., Nakamura H., Subr V., Ulbrich K. and Maeda H. (2015) Photodynamic therapy and imaging based on tumor-targeted nanoprobe, polymer-conjugated zinc protoporphyrin. Future Science OA 1:3.
Grimes, D. R. and M. Partridge, A mechanistic investigation of the oxygen fixation hypothesis and oxygen enhancement ratio. Biomed Phys Eng Express, 2015. 1(4): p. 045209.
Hrkach, J. et al. Preclinical development and clinical translation of a PSMA-targeted docetaxel nanoparticle with a differentiated pharmacological profile. Sci Transl Med, 2012. 4(128): p. 128ra39.
Jianshe, W. et al. One-step synthesis of highly water-soluble LaF3:Ln3+nanocrystals in methanol without using any ligands. Nanotechnology, 2007. 18(46): p. 465606.
Liu, Y. et al. Investigation of water-soluble x-ray luminescence nanoparticles for photodynamic activation. Applied Physics Letters, 2008. 92(4): p. 043901.
Ma, L., X. Zou, and W. Chen. A new X-ray activated nanoparticle photosensitizer for cancer treatment. J Biomed Nanotechnol, 2014. 10(8): p. 1501-8.
Moore, R. B. et al. Measurement of PDT-induced hypoxia in Dunning prostate tumors by iodine-123-iodoazomycin arabinoside. J Nucl Med, 1993. 34(3): p. 405-11.
Moore, R. B. et al. A comparison of susceptibility to photodynamic treatment between endothelial and tumor cells in vitro and in vivo. Photodiagnosis Photodyn Ther, 2007. 4(3): p. 160-9.
Retif, P. et al. Nanoparticles for Radiation Therapy Enhancement: the Key Parameters. Theranostics, 2015. 5(9): p. 1030-44.
Rockwell, S. et al. Hypoxia and radiation therapy: Past history, ongoing research, and future promise. Curr Mol Med, 2009. 9(4): p. 442-58.
Shi J., Wang L., Gao J., Liu Y., Zhang J., Ma R., Liu R. and Zhang Z. (2014) A fullerene-based multi-functional nanoplatform for cancer theranostic applications. Biomaterials. 35(22): 5771-5784.
Takahashi, J. and M. Misawa, Analysis of Potential Radiosensitizing Materials for X-Ray-Induced Photodynamic Therapy. NanoBiotechnology, 2007. 3(2): p. 116-126.
Tang Y., Hu J., Elmenoufy A. H. and Yang X. (2015) Highly efficient FRET system capable of deep photodynamic therapy established on x-ray excited mesoporous LaF3:Tb scintillating nanoparticles. ACS Appl Mater Interfaces. 7:12261-12269.
Thakor, A. S. and S. S. Gambhir, Nanooncology: the future of cancer diagnosis and therapy. CA Cancer J Clin, 2013. 63(6): p. 395-418.
Wilson, B. C. and M. S. Patterson, The physics, biophysics and technology of photodynamic therapy. Phys Med Biol, 2008. 53(9): p. R61-109.
Xiao, Z., et al., Fractionated versus Standard Continuous Light Delivery in Interstitial Photodynamic Therapy of Dunning Prostate Carcinomas. Clinical Cancer Research, 2007. 13(24): p. 7496.
Yun, J., et al., Evaluation of a lung tumor autocontouring algorithm for intrafractional tumor tracking using low-field MRI: a phantom study. Med Phys, 2012. 39(3): p. 1481-94.
Zou X., Yao M., Ma L., Hossu M., Han X., Juzenas P. and Chen W. (2014) X-ray-induced nanoparticle-based photodynamic therapy of cancer. Nanomedicine (Lond). October; 9(15):2339-51.
Zhu, T. C., et al., Macroscopic Modeling of the singlet oxygen production during PDT. Proc SPIE Int Soc Opt Eng, 2007. 6427: p. 642708.

What is claimed is:

1. A nanoparticle comprising a nanocarrier encapsulating a nanoscintillator capable of emitting light upon exposure to radiation; a photosensitizer capable of absorbing the light from the nanoscintillator to generate singlet oxygen species; and optionally, one or more diagnostic agents, therapeutic agents, or a combination thereof.

2. The nanoparticle of claim 1, wherein the nanocarrier comprises a hydrophobic compound and a hydrophilic compound.

3. The nanoparticle of claim 2, wherein the hydrophobic compound comprises poly lactide co-glycolide acid.

4. The nanoparticle of claim 3, wherein the hydrophilic compound comprises polyethylene glycol, or polyethylene glycol bound to an antibody.

5. The nanoparticle of claim 1, wherein the nanoscintillator comprises a radioluminescent material doped with a dopant selected from terbinium, yttrium, sodium, or cerium.

6. The nanoparticle of claim 5, wherein the amount of dopant ranges from about 0% to about 50%.

7. The nanoparticle of claim 5, wherein the nanoscintillator comprises lanthanum fluoride doped with 10% cerium.

8. The nanoparticle of claim 5, wherein the nanoscintillator is hydrophobic.

9. The nanoparticle of claim 5, wherein the nanoscintillator is operably associated with the photosensitizer by physical proximity or chemical linkage.

10. The nanoparticle of claim 1, wherein the photosensitizer is selected from a thiocyanate, vertiporfin, hypocrellin A, hypocrellin B, or protoporphyrin IX.

11. The nanoparticle of claim 10, wherein the photosensitizer is hydrophobic.

12. The nanoparticle of claim 1, having a size ranging from about 30 nm to about 150 nm.

13. The nanoparticle of claim 12, having a size ranging from about 100 nm to about 120 nm.

14. A pharmaceutical composition comprising the nanoparticle of any one of claims 1-13 and a pharmaceutically acceptable carrier.

15. A method of treating, preventing, or ameliorating a disease in a subject, comprising the steps of:
a) administering to the subject an effective amount of the nanoparticle of claim 1, or the pharmaceutical composition of claim 14; and
b) applying radiation to the subject.

16. The method of claim 15, wherein the nanoparticle or the pharmaceutical composition is administered to the subject intravenously.

17. The method of claim 15, wherein the radiation comprises X-rays.

18. The method of claim 15, wherein the disease is selected from cancer, a dermatological condition, an infection, macular degeneration, Barrett's esophagus, a deep abdominal abscess, or a condition requiring cell cytotoxicity.

19. The method of claim 15, wherein the nanoparticle comprises one or more diagnostic agents, therapeutic agents, or a combination thereof for release in a localized area following application of the radiation.

20. Use of the nanoparticle of claim 1, or the pharmaceutical composition of claim 14 to treat, prevent, or ameliorate a disease in a subject.
